(19) United States
(12) Patent Application Publication
(10) Pub. No.: US 2019/0178887 A1
(43) Pub. Date: Jun. 13, 2019

(54) Method for diagnosing a fungal infection

(71) Applicant: EUROIMMUN Medizinische Labordiagnostika AG, Luebeck (DE)

(72) Inventors: Victor HERBST, Krummesse (DE); Kathrin HOFFMANN, Luebeck (DE)

(73) Assignee: EUROIMMUN Medizinische Labordiagnostika AG, Luebeck (DE)

(21) Appl. No.: 16/208,758

(22) Filed: Dec. 4, 2018

(30) Foreign Application Priority Data
Dec. 11, 2017 (EP) 17206386.9

(51) Int. Cl. G01N 33/569; G01N 33/577

(52) U.S. Cl. CPC G01N 33/56961; G01N 2333/38; G01N 2800/26; G01N 33/577

(57) ABSTRACT

An Aspergillus infection is diagnosed in a patient by detecting in a sample a proteinaceous antigen derived from mycelium of a pathogenic Aspergillus strain, wherein the sample is contacted with a first antibody binding specifically to said antigen and wherein any antigen bound to said first antibody is detected by way of a non-membrane based immunoassay, preferably selected from an ELISA and a bead-based assay. An antibody binding to a proteinaceous antigen derived from mycelium of a pathogenic Aspergillus strain is used for increasing the reliability of an immunoassay or the diagnosis of an Aspergillus infection in a patient. A further method includes coating a diagnostic device with a first antibody binding specifically to a proteinaceous antigen derived from mycelium of a pathogenic Aspergillus strain.

### CROSS-REFERENCE TO RELATED APPLICATIONS

This application claims priority to European patent application 17206386.9 filed Dec. 11, 2017, incorporated herein by reference.

### BACKGROUND OF THE INVENTION

The present invention relates to a method for diagnosing an Aspergillus infection in a patient, comprising the step detecting in a sample beta-1,5-galactofuranose from mycelium of a pathogenic Aspergillus strain, wherein the sample is contacted with a first antibody binding specifically to said antigen and wherein any antigen bound to said first antibody is detected by way of a non-membrane based immunoassay, preferably selected from the group comprising an ELISA and a bead-based assay and a use of an antibody binding to beta-1,5-galactofuranose from mycelium of a pathogenic Aspergillus strain for increasing the reliability of an immunoassay for the diagnosis of an Aspergillus infection in a patient as well as the method comprising the step coating a diagnostic device with a first antibody binding specifically to beta-1,5-galactofuranose from mycelium of a pathogenic Aspergillus strain.

Invasive aspergillosis (IA) is an important cause of morbidity and mortality in patients with haematological malignancies and profound immuno-suppression, for example after haematopoietic stem cell transplantation, but also non-haematological patients, for example those suffering from a condition selected from the group comprising diabetes mellitus, systemic lupus erythematosus, old pulmonary tuberculosis and transplantation of liver or kidney.

Rapid detection of IA using immunodiagnostics has centred around the detection of fungal galactomannan (GM) (Latge et al. (1994) Chemical and immunological characterization of the extracellular galactomannan secreted by Aspergillus fumigatus. Infect. Immun. 62:5424-5433; Pazos, et al. (2005) Contribution of (1˜3)-ß-D-glucan chromogenic assay to diagnosis and therapeutic monitoring of invasive aspergillosis in neutropenic adult patients: a comparison with serial screening for circulating galactomannan. J. Clin. Microbiol. 43:299-305; Quindos (2006) New microbiological techniques for the diagnosis of invasive mycoses caused by filamentous fungi. Clin. Microbiol. Infect. 12:40-52). GM is a branched carbohydrate comprising a mannose main chain and short side chains comprising a galactose molecule.

Monoclonal antibodies (mAbs) have been successfully used in the detection of GM, and they form the basis of commercial laboratory based tests such as the Platelia Aspergillus ELISA kit that incorporates a rat mAb (EB-A2) directed against tetra (1˜5)-ß-D galactofuranoside, the immunodominant epitope in the antigen (Morelle et al. (2005) Galactomannoproteins of Aspergillus fumigatus. Euk. Cell 4: 1308-1316; Stynen et al. (1995) A new sensitive sandwich enzyme linked immunosorbent assay to detect galactofuran in patients with invasive aspergillosis. J. Clin. Microbiol. 33:497-500; Stynen et al. (1992) Rat monoclonal antibodies against Aspergillus galactomannan. Inf. Immun. 60:2237-2245).

Immunoassays for GM detection are a significant asset for managing patients at risk of catching IA because of detection of the antigen in the early stages of disease progression. Despite their widespread use, recent studies have revealed significant variation in performance.

False positive results have been attributed to cross-reaction of mAb EB-A2 with various reagents such as GM from non-Aspergillus fungi (Giacchino et al. (2006) Aspergillus galactomannan enzyme-linked immunosorbent assay cross-reactivity caused by invasive Geotrichum capitatum. J. Clin. Microbiol. 44:3432-3434; Kappe et al. (1993) New cause for false positive results with the Pastorex Aspergillus antigen latex agglutination test. J. Clin. Microbiol. 31:2489-2490, Quindos (2006) New microbiological techniques for the diagnosis of invasive mycoses caused by filamentous fungi. Clin. Microbiol. Infect. 12:40-52; Swanink et al. (1997) Specificity of the sandwich enzyme-linked immunosorbent assay for detecting Aspergillus galactomannan. J. Clin. Microbiol. 35:257-260; Verweij et al. (2006) Issues with galactomannan testing. Med. Mycol. 44: 179 183), and galactoxylomannan from Cryptococcus neoformans; Dalle et al. (2005) Cryptococcus neoformans galactoxylomannan contains an epitope(s) that is cross-reactive with Aspergillus galactomannan. J. Clin. Microbiol. 43:2929-2931.).

Whilst several authors have reported satisfactory sensitivities of >85%, sensitivity of the assay can vary considerably and was reported as low as 29% (Verweij et al. (2006) Issues with galactomannan testing. Med. Mycol. 44: 179-183). Several lines of evidence suggest that the sensitivity very much depends on the cohort of patients chosen and the way they have been treated. For example, lower sensitivities have been reported with samples from patients treated with posaconazol (Marr et al. (2005). Antifungal Therapy Decreases Sensitivity of the Aspergillus Galactomannan Enzyme Immunoassay. Clin Infect Dis. 40, 1762-1769.) The reason is unknown. It seems that many authors reporting higher sensitivities used serum samples from cohorts excluding such patients.

Another immunoassay was developed by Thornton (2008) (Development of an Immunochromatographic Lateral-Flow Device for Rapid Serodiganosis of Invasive Aspergillosis, Clin. And Vacc. Immun. 15-7, 1095-1105). It is based on the use of the monoclonal antibody JF5 to the Aspergillus JF5 antigen in a Lateral Flow Device, which was later identified as beta-1,5-galactofuranose. However, such devices are hardly useful for the high-throughput screening of samples in routine diagnostics. Furthermore, the sensitivity of this assay, according to this study, leaves significant room for improvement, as a range of false-negative results were obtained.

The same assay has been disclosed in WO2010/082034, incorporated herein in its entirety by reference, but both the GM ELISA and the JF5 Lateral Flow Device assay were reported to have essentially the same sensitivity (100%). Since samples from artificially infected Guinea pigs were used, these results cannot be translated to humans, and the fact remains that there remains room for improving the sensitivities of the assays for use in the diagnosis of humans. WO2010/082034 discloses an ELISA assay, but only for checking the quality of antibody using coated antigen, not a diagnostic assay based on human samples, let alone a boiling step.

Consistent with this, when Hoenigl et al. (2014) (Performance of Galactomannan, Beta-D-Glucan, Aspergillus Lateral-Flow-Device, Conventional Culture and PCR Tests with Bronchoalveolar Lavage Fluid for Diagnosis of Invasive Pulmonary Aspergillosis, J. Clin. Microbiol 52-6, 2039-2045) compared these methods using human samples, significantly lower sensitivities were reported, more specifically 80% for both assays.

Davies et al. (2017) (Towards translational ImmunoPET/MR Imaging of Invasive Pulmonary Aspergillosis: The Humanised Monoclonal Antibody JF5 Detects Aspergillus Lung Infections In Vivo, Theranostics, 7-4, 3398-3414, incorporated herein in its entirety by reference) reported the use of humanized monoclonal antibody hJF5 for the in vivo detection of Aspergillus infections. As part of their investigations, they used monoclonal and humanized JF5 antibodies to detect Aspergillus infections in murine, but not human samples. Besides, murine lung tissue obtained by excision of the lungs were examined. The diagnostic reliability of the antibodies for routine high-throughput screening of human samples was not addressed. It is not disclosed that the samples were subjected to boiling.

Ku et al. (2012) report a rather unsatisfactory performance of the GMA ELISA with samples from non-haematological patients. The sensitivity was 23.1%, a value considered “very low” by the authors.

Therefore, a problem underlying the present invention is to provide an improved non-membrane-based immunoassay, preferably selected from the group comprising ELISA or bead-based assay, that overcomes shortcomings of conventional assays, in particular lack of reproducibility, specificity and sensitivity, and may preferably be used for high-throughput screening of samples.

Another problem underlying the present invention is to overcome shortcomings of conventional assays, in particular increasing specificity and sensitivity, with regard to patients suffering from CGD (chronic granulomatous disease) and/or hyper-IgE syndrome with recurrent infection (HIES; Job syndrome).

Another problem underlying the present invention is to provide a diagnostically reliable assay, in particular having optimized specificity and/or sensitivity, that may be used in a high-throughput format for a multitude of samples in an automated manner.

Another problem underlying the present invention is to provide a diagnostically reliable assay for high-throughput screening having an improved sensitivity when used to test samples from patients treated with anti-fungal drugs such as posaconazole or non-haematooncological patients.

Another problem underlying the present invention is to provide a diagnostically reliable assay, wherein a positive diagnosis can be accomplished at an early stage of the infection and/or a late stage of the infection, in particular earlier or later, respectively compared to state of the art assays, leading to a an extended period in which a reliable diagnosis based on a serological assay is possible.

### BRIEF SUMMARY OF THE INVENTION

The problem underlying the present invention is solved by the subject matter of the embodiments of the present invention which include:

1. A method for diagnosing an Aspergillus infection in a patient, comprising the step detecting in a sample beta-1,5-galactofuranose from mycelium of a pathogenic Aspergillus strain,

wherein the sample is contacted with a first antibody binding specifically to said antigen, and
wherein any antigen bound to said first antibody is detected by way of a non-membrane based immunoassay, preferably selected from the group comprising an ELISA and a bead-based assay.

2. A use of an antibody binding specifically to beta-1,5-galactofuranose from mycelium of a pathogenic Aspergillus strain for increasing the diagnostic reliability of an immunoassay for the diagnosis of an Aspergillus infection in a patient comprising the step detecting said antigen in a sample,

wherein the sample is contacted with a first antibody to said antigen followed by detection of any antigen bound to said first antibody by way of a non-membrane based immunoassay, preferably selected from the group comprising an ELISA and a bead-based assay.

3. The method or use according to any of 1 to 2, wherein the immunoassay is an ELISA, preferably a sandwich ELISA.

4. A method comprising the step coating a diagnostic device with a first antibody binding specifically to beta-1,5-galactofuranose from mycelium of a pathogenic Aspergillus strain,

wherein the diagnostic device is a non-membrane based device, preferably selected from the group comprising an ELISA microplate and a bead, preferably a microplate.

5. The method or use according to any of 1 to 4, wherein the first antibody is coated on a microplate or a bead.

6. The method or use according to any of 1 to 5, wherein the first antibody is a monoclonal antibody.

7. The method or use according to any of 1 to 6, wherein the first antibody is the antibody JF5.

8. The method or use according to any of 1 to 7, wherein the Aspergillus strain is selected from the group comprising A. fumigatus, A. flavus, A. niger, A. terreus and A. nidulans and is preferably A. fumigatus AF293.

9. The method or use according to any of 1 to 8, wherein the sample is selected from the group comprising a serum sample and a bronchoalveolar lavage sample, preferably from a human patient.

10. The method or use according to any of 1 to 9, wherein the method is for diagnosing an invasive aspergillosis.

11. The method or use according to any of 1 to 10, wherein the sample is subjected to a denaturing step prior to contacting the sample with the antibody.

12. The method or use according to 11, wherein the denaturing step is a boiling step.

13. The method or use according to any of 1 to 12, wherein the antigen is detected using a second antibody, which is preferably the same antibody as the first antibody.

14. The use according to any of 2 to 13, wherein the use is for increasing the specificity, preferably in the diagnosis of a human patient treated with an anti-fungal drug, more preferably Posaconazole.

15. The use according to any of 2 to 14, wherein the use is for increasing the diagnostic reliability at an early stage of the infection.

### DETAILED DESCRIPTION OF THE INVENTION

In a first aspect, the problem underlying the present invention is solved by a method for diagnosing an Aspergillus infection in a patient, comprising the step detecting in a sample beta-1,5-galactofuranose from mycelium of a pathogenic Aspergillus strain, wherein the sample is contacted with a first antibody binding specifically to beta-1,5-galactofuranose and wherein any antigen bound to said first antibody is detected preferably by way of a non-membrane-based immunoassay, more preferably selected from the group comprising a an ELISA and a bead-based assay.

In a second aspect, the problem underlying the present invention is solved by a use of an antibody binding specifically to beta-1,5-galactofuranose from mycelium of a pathogenic Aspergillus strain for increasing the diagnostic reliability of an immunoassay for the diagnosis of an Aspergillus infection in a patient comprising the step detecting said antigen in a sample, wherein the sample is contacted with a first antibody to said antigen followed by detection of any antigen bound to said first antibody preferably by way of a membrane-based immunoassay, more preferably selected from the group comprising a an ELISA and a bead-based assay.

In a preferred embodiment the immunoassay is an ELISA, preferably a sandwich ELISA.

In a 3^{rd} aspect, the problem underlying the present invention is solved by a method comprising the step coating a diagnostic device with a first antibody binding specifically beta-1,5-galactofuranose from mycelium of a pathogenic Aspergillus strain, wherein the diagnostic device is preferably a non-membrane based device, more preferably selected from the group comprising a microplate and a bead, preferably a microplate.

In a preferred embodiment, the first antibody is coated on a microplate or a bead.

In a preferred embodiment, the first antibody is a monoclonal antibody.

In a first embodiment the antibody is the antibody JF5 or a variant thereof.

In a preferred embodiment, the Aspergillus strain is selected from the group comprising A. fumigatus, A. flavus, A. niger, A. terreus, A. nidulans and is preferably A. fumigatus AF293.

In a preferred embodiment, the sample is selected from the group comprising a serum sample and a bronchoalveolar lavage sample.

In a preferred embodiment, the method is for diagnosing an invasive aspergillosis.

In a preferred embodiment, the sample is subjected to a denaturing step, preferably a boiling step prior to contacting the sample with the antibody.

In a preferred embodiment, the antigen is detected using a second antibody, which is preferably the same antibody as the first antibody.

In a preferred embodiment, the use is for increasing the sensitivity.

In a preferred embodiment, the use is for increasing the diagnostic reliability, preferably sensitivity or specificity, at an early stage of the infection.

The present invention is based on the inventors' surprising finding that high-throughput assays not based on the use of membranes such as an ELISA for the detection beta-1,5-galactofuranose from mycelium of a pathogenic Aspergillus strain such as the JF5 antigen have a significantly increased sensitivity compared to conventional similar tests based on other methods of detection. By contrast, the state of the art suggests that membrane-based immunoassays based on JF5 antigen, exemplified by lateral flow device, have the same sensitivity as the immunoassay based on the galactomannan antigen.

Moreover, the state of the art suggests that the conventional ELISA based on galactomannan detection, when compared to other immunoassays, provides optimal sensitivity that leaves no room for improvement. The invention is based on the inventors' surprising finding that the sensitivity may be improved in patient cohorts comprising patients treated with anti-fungal drugs such as posaconazole if an ELISA for the detection of proteinaceous antigen derived from mycelium of a pathogenic Aspergillus strain such as JF5 is used.

The present invention is also based on the inventors' surprising finding that denaturing a sample, for example by way of boiling, increases the diagnostic reliability of the test, in particular the specificity at low concentrations including the absence of the antigen such that false positive results may be avoided. The authors theorize that this releases the antigen which may otherwise be masked by other components or compete for binding with other components in the samples or denatures other components in the sample having unwanted cross-reactivity which may lead to false positive reactions.

In a preferred embodiment, the antibody used binds to beta-1,5-galactofuranose. This may be purified beta-1,5-galactofuranose, but it is preferably beta-1,5-galactofuranose from the mycelium of an infectious Aspergillus strain, where it may be associated with larger macromolecules such as carbohydrates carrying more than one molecule of beta-1,5-galactofuranose. Such antigenic beta-1,5-galactofuranose from mycelium may be isolated as disclosed for what is referred to as “JF5 antigen” in WO2010082034.

The preparation of such antigens and the generation of diagnostically useful antibodies that may be used as the first antibody to detect them has been described in detail in WO2010082034, Example 1. Briefly, mice were immunized with lyophilized mycelium of a pathogenic Aspergillus strain suspended in PBS, followed by standard production of hybridoma and determination of antibody specificity by ELISA using supernatant of a range of hybridoma.

In a preferred embodiment, a sample selected from the group comprising a serum sample and a bronchioalveolar lavage sample, preferably from a mammalian, more preferably human patient, is used for the assay. The patient may be a patient suspected of suffering from IA. Preferably, the patient is a non-haematological patient or a patient treated with anti-fungal drugs such as posaconazole. Non-haematological patients may include patients having undergone transplantation of solid organs, for example lung, liver or kidney, patients suffering from polytrauma, patients under intensive care and patients suffering from non-haematological tumors.

At least one first antibody binding to said antigen is used according to the present invention. The JF5 antibody disclosed in WO2010082034 may be used or, based on its sequences disclosed in WO2010082034 and in Davies et al. (2017), the person skilled in the art can readily design and purify variants of said antibody that also bind to beta-1,5-galactofuranose from mycelium of a pathogenic Aspergillus strain, preferably A. fumigatus. In a preferred embodiment, the antibody and variants thereof binds specifically to beta-1,5-galactofuranose from Aspergillus, preferably A. fumigatus. The antibody hjF5 disclosed in Davies et al. and variants thereof may also be used. The binding specificity of such an antibody obtained may be confirmed by repeating the experiments in WO2010082034, more specifically Example 3. Briefly, Hartley guinea pigs may be infected by way of exposure to conidia made from a pathogenic Aspergillus strain, followed by an immunoassay using the antibody to be characterized.

A second antibody binding to said antigen may be used in alternative assay formats involving the use of the first antibody for immobilizing the antigen and the second antibody for detecting the immobilized antigen. Preferably the antigen is an antigen carrying more than one molecule of beta-1,5-galactofuranose such that both a first antibody and a second antibody binding to beta-1,5-galactofuranose may bind to the antigen at the same time.

In a preferred embodiment, the first and/or second antibody is an antibody binding specifically to the antigen or epitope recognized. In a preferred embodiment, the term “binding specifically”, as used herein, means that the binding is stronger than a binding reaction characterized by a dissociation constant of 1×10⁻⁵ M, more preferably 1×10⁻⁷ M, more preferably 1×10⁻⁸ M, more preferably 1×10⁻⁹ M, more preferably 1×10⁻¹⁰ M, more preferably 1×10⁻¹¹ M, more preferably 1×10⁻¹² M, as determined by surface plasmon resonance using Biacore equipment at 25° C. in PBS buffer at pH 7.

In a preferred embodiment, the immunoassay is a non-membrane based immunoassay. In a more preferred embodiment, the term “non-membrane based immunoassay”, as used herein, refers to an immunoassay carried out in the absence of a membrane, in particular a membrane on which a reagent such as an antibody or an antigen is immobilized. Examples of membrane-based immunoassays include lateral flow device, western blot, dot blot and line blot. Examples of non-membrane based immunoassays include bead-based immunoassays and ELISAs based on the use of a microtiter plate.

In a preferred embodiment, the diagnostic device or carrier is a bead. In a preferred embodiment, the term “bead” as used herein, refers to a solid bead, preferably made from a carbohydrate or derivate thereof such as agarose or sepharose, to which an antigen or antibody is attached, preferably in a covalent manner. If a bead is used as diagnostic device, it is preferred that the antigen or second antibody has a chemiluminescent label and is detected by chemiluminescence. In a preferred embodiment, the bead is a magnetic bead. Again, the antigen is preferably an antigen carrying more than one molecule of beta-1,5-galactofuranose such that both a first antibody and a second antibody binding to beta-1,5-galactofuranose may bind to the antigen at the same time.

A first antibody binding specifically to beta-1,5-galactofuranose from mycelium of a pathogenic Aspergillus strain and optionally, for example in a ELISA immunoassay based on the use of a first antibody for immobilizing the antigen, a second antibody binding specifically to beta-1,5-galactofuranose from mycelium of a pathogenic Aspergillus strain may be used according to the present invention such as the antibody JF5 or the antibody AB633-HC3-LC2-T1-1B3-1G10 (hjF5) published by Davies et al. (2017) or variants thereof.

In a preferred embodiment, the term “antibody” as used herein, is to be interpreted broadly and comprises not only full-length antibodies but also derivatives and fragments thereof such as Fab, F(ab′)2, Fr, ScTv, Fdb and dAb.

In another preferred embodiment, the term “variant” of an antibody, as used herein, refers to a polypeptide or a fragment of said antibody comprising amino acid sequences, preferably a fragment comprising at least 80, more preferably 100, more preferably 110 successive amino acids, that are at least 40, 50, 60, 70, 75, 80, 85, 90, 92, 94, 95, 96, 97, 98 or 99% identical to the reference amino acid sequence referred to or the fragment thereof, wherein amino acids other than those essential for the biological activity, for example the ability to bind specifically to an antigen of interest, or the fold or structure of the polypeptide are deleted or substituted and/or one or more such essential amino acids are replaced in a conservative manner and/or amino acids are added or deleted such that the biological activity of the polypeptide is at least partially preserved.

The state of the art comprises various methods that may be used to align two given nucleic acid or amino acid sequences and to calculate the degree of identity, see for example Arthur Lesk (2008), Introduction to bioinformatics, Oxford University Press, 2008, 3^{rd} edition. In a preferred embodiment, the ClustalW software (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007): Clustal W and Clustal X version 2.0. Bioinformatics, 23, 2947-2948) is used applying default settings.

In a preferred embodiment, variants may, in addition, comprise chemical modifications, for example labels or covalent modifications such as glycosylation, phosphorylation, acetylation, decarboxylation, citrullination, hydroxylation and the like. The person skilled in the art is familiar with methods for the modification of polypeptides. Moreover, variants may also be generated by way of fusion with other known polypeptides or variants thereof.

The variant of the antibody has biological activity. In a preferred embodiment such biological activity is the ability to bind specifically to beta-1,5-galactofuranose, preferably beta-1,5-galactofuranose from mycelium of a pathogenic Aspergillus strain, most preferably from A. fumigatus, which is preferably an antigen secreted exclusively during active growth. Whether or not a variant of the antibody has biological activity may be readily determined in an ELISA as described in Example 1 of the present invention.

In a preferred embodiment, the immunoassay is an ELISA carried out using a microtiter plate. In a preferred embodiment, the microtiter plate is contacted with sample comprising the antigen to be detected, followed by detection of the antigen using a first antibody. In another preferred embodiment, the microtiter plate is coated with a first antibody, followed by incubation with sample comprising antigen and detection of any antigen bound to the first antibody using a second antibody. The second antibody may carry a detectable label or may be detected using a conjugated antibody recognizing the second antibody, and the conjugated antibody carries a detectable label or detected indirectly by binding of a label. In a preferred embodiment, the detectable label is selected from the group comprising a fluorescent, chemiluminescent, radioactive or enzymatically active label.

In a preferred embodiment, the same antibody is used as the first and the second antibody.

In another preferred embodiment, the microtiter plate is coated with an antibody, then contacted with the sample comprising the antigen to be detected, followed by incubation in the presence of antigen comprising a detectable label which then competes with the antigen to be detected.

In another embodiment, the microtiter plate is coated with the antigen, then contacted with the sample comprising the antigen to be detected. In a following incubation step these both then will compete for the binding sites of an added labelled antibody.

The inventive teachings provide a kit, preferably for diagnosing aspergillosis. Such a kit is a container that comprises specific reagents required to practice the inventive method, in particular a non-membrane based carrier, preferably selected from the group comprising an ELISA microtiter plate and a bead, coated with antigen, preferably in the form of antigen immobilized via a first antibody binding specifically to the antigen, or a first antibody binding specifically to a proteinaceous antigen derived from mycelium of a pathogenic Aspergillus strain. Any antigen coated may be antigen carrying a detectable label. Optionally the kit comprises to one or more solutions required to practice the inventive method in addition. The solution is or the solutions are preferably selected from or are all solutions from the group comprising sample dilution buffer, washing buffer and buffer comprising a means for detecting a bound antibody, such as a secondary antibody and optionally a means for detecting the latter. The kit may comprise a second antibody binding specifically to the antigen, which second antibody is in a solution or in a solid form made to prepare such a solution. In a preferred embodiment, the first and the second antibody is the same antibody, and it is in a more preferred embodiment the antibody binding to the JF5 antigen. The kit may comprise at least three, four or five different solutions having different, known concentrations of beta-1,5-galactofuranose or an Aspergillus cell or a derivative thereof for setting up a calibration curve or as a positive or negative control. Furthermore, the kit may comprise instructions detailing how to use the kit and the inventive diagnostically useful carrier.

In a preferred embodiment, the term “diagnosis”, as used herein, refers to any kind of procedure aiming to obtain information instrumental in the assessment whether a patient suffers or is likely or more likely than the average or a comparative subject, the latter preferably having similar symptoms, to suffer from IA in the past, at the time of the diagnosis or in the future, to find out how the disease is progressing or is likely to progress in the future or to evaluate the responsiveness of a patient with regard to a certain treatment, for example the administration of suitable drugs such as antifungal drugs. In other words, the term “diagnosis” comprises not only diagnosing, but also prognosticating and/or monitoring the course of a disease or disorder.

Therefore, the term “diagnosis” does preferably not imply that the diagnostic methods or agents according to the present invention will be definitive and sufficient to finalize the diagnosis on the basis of a single test, let alone parameter, but may refer to a contribution to what is referred to as a “differential diagnosis”, i.e. a systematic diagnostic procedure considering the likelihood of a range of possible conditions on the basis of a range of diagnostic parameters. The term “diagnosis” may also refer to a method or agent used to choose the most promising treatment regimen for a patient. In other words, the method or agent may relate to selecting a treatment regimen for a subject.

The invention provides a use of an antibody binding specifically to beta-1,5-galactofuranose from mycelium of a pathogenic Aspergillus strain or a non-membrane based carrier, preferably an ELISA microplate or a bead, coated with said antibody for the manufacture of a kit for the diagnosis of IA, in which a diagnostic assay with an increased sensitivity is provided. Such manufacture may relate to a method comprising the step immobilizing on a diagnostically useful carrier an antibody binding specifically to a proteinaceous antigen derived from mycelium of a pathogenic Aspergillus strain, wherein the carrier is a non-membrane based carrier, preferably selected from the group comprising an ELISA microtiter plate and a bead.

In a preferred embodiment, the term “diagnostic reliability”, as used herein refers to the combination of sensitivity and specificity of an immunoassay.

In a preferred embodiment, the term “sensitivity”, as used herein, refers to the ratio of positive samples correctly recognized as such by carrying out the assay of interest and the total number of samples examined. In other words, an assay has 100% sensitivity if all samples from infected patients give a positive result, i.e. there are no false negative results.

In a preferred embodiment, the term “specificity”, as used herein, refers to the ratio of negative samples correctly recognized as such by carrying out the assay of interest and the total number of samples examined. In other words, an assay has 100% specificity if all samples from healthy subjects give a negative result, i.e. there are no false positive results.

In a preferred embodiment, the sample is subjected to a denaturing step, preferably a boiling step prior to the step contacting the sample with the antibody. A chelating reagent such as EDTA may be present. This may be accomplished by elevating the temperature in the sample such that the liquid sample solution starts boiling, for example by placing the sample in a boiling water bath. In a more preferred embodiment, the sample is kept for at least 1, 2, 3, 4, 5 or 10 minutes, preferably 3 minutes at a temperature that will cause the sample to boil, for example 100° C. Subsequently, any insoluble components may be separated from the liquid sample, preferably by subjecting the boiled sample to centrifugation. The supernatant is then used for the next steps as the sample for the immunoassay. Alternatively, the denaturing step may be accomplished by adding a denaturing agent, for example 6 molar guanidinium hydrochloride, which must, however, be removed or its concentration must be reduced such that the binding activity antibodies used in the method according the present invention is maintained.

In a preferred embodiment, the present invention provides a use of an antibody binding specifically to beta-1,5-galactofuranose or of a nucleic acid encoding an antibody binding specifically to beta-1,5-galactofuranose or a nucleic acid hybridizing specifically to a nucleic acid encoding an antibody binding specifically to beta-1,5-galactofuranose or a vector or cell comprising said nucleic acid for the manufacture of kit for the diagnosis of a an Aspergillus infection at an increased diagnostic reliability.

In another preferred embodiment, the method may be an in vitro method for determining or confirming the reliability of an antibody detection assay and may involve detecting an beta-1,5-galactofuranose or an Aspergillus cell in a solution, which is not a sample from a patient, but is known to comprise beta-1,5-galactofuranose or an Aspergillus cell or a derivative thereof, preferably at a known concentration. In a more preferred embodiment, the method and reagents may be used for setting up a calibration curve comprising at least two, preferably at least three, four or five different points representing different concentrations of beta-1,5-galactofuranose or an Aspergillus cell or a derivative thereof, wherein preferably one solution comprising a known concentration is tested in a separate assay for each of the at least three, four or five different points. Such a solution comprising a known concentration is preferably not a sample derived from the human body. Alternatively, the solution may be a negative control not comprising the antigen to check the background. Such method may be run in parallel with, after or before a diagnostic method.

In another preferred embodiment, the present invention provides a use of a denatured sample, preferably serum sample, for increasing the diagnostic reliability, preferably specificity of a for diagnosing an Aspergillus infection in a patient, preferably comprising the step detecting in the sample beta-1,5-galactofuranose from mycelium of a pathogenic Aspergillus strain,

wherein the sample is contacted with a first antibody binding specifically to said antigen and
wherein preferably any antigen bound to said first antibody is detected by way of a non-membrane based immunoassay, preferably selected from the group comprising an ELISA and a bead-based assay. The first antibody may be present in the sample when it is used.

### EXAMPLES

### Example 1

1. Patients:

A cohort comprising 38 Patients suffering from confirmed IA was recruited.

Patients were diagnosed by expert clinicians based on the EORTC/MSG guidelines. They included a wide spectrum of subjects comprising both haematooncologic patients and patients under intensive care. A range of non-infectious patients were used as controls.

From each patient, several samples taken at various time points were measured to exclude the possibility that antibody titers were too low for detection as a result of the disease being at an early stage.

Blood samples from a control cohort of 79 healthy subjects suspected of suffering from a tick-borne disease were tested as a negative control.

2. Preparation of Sample

300 μl of a sample or calibrator (such as human serum spiked with varying amounts of A. fumigatus antigen as in Example 3) were mixed using 100 μl sample diluent (PBS comprising 0.1M EDTA). This mixture was then heated for 3 minutes in a boiling water bath, spun down (10 minutes at 10.000×g), followed by recovering the supernatant, which was subsequently used.

3. Preparation of Antibody-Coated Wells

JF5 antibody described in WO2010/082034 was immobilized on the surface of wells from a microplate (NUNC MaxiSorp) using standard methods.

4. Assay

100 μl of said mixture comprising calibrators, controls or sample supernatant were transferred in a microplate well coated with JF5 antibodies

For the incubation step, wells were covered and kept for one hour at room temperature (18° C. to 25° C.) at medium speed (approximately 450 rpm) on an orbital shaker. Samples were removed, followed by addition of 300 μl per well of washing buffer (available as ZE1120 from EUROIMMUN AG, Lubeck) and an incubation for 30 to 60 seconds per washing cycle. Three washing cycles were carried out.

Washing buffer was removed, followed by addition of 100 μl per well of biotinylated JF5 antibody in EUROIMMUN Probenpuffer (Catalogue number ZE1100). Wells were covered and incubated at room temperature for one hour on an orbital shaker (450 rpm). The washing steps were repeated.

In a third incubation step 100 μl Enzyme conjugate (EUROIMMUN AG, ZE 6911-2) is added and incubated 30 min at room temperature on an orbital shaker (450 rpm).

Washing steps were repeated, followed by addition of 100 μl per well of chromogen and substrate (Standard ELISA TMB substrate, available from EUROIMMUN AG, catalogue number ZE 1200) and incubation for 15 minutes at room temperature. The reaction was stopped by addition of 100 μl per well of stop solution (EUROIMMUN AG, catalogue number Cat# ZE 1210).

Absorbance of the solution was measured at 450 nm.

Samples were determined as positive if the absorbance of 450 nm was beyond a cut-off threshold calculated by routine methods.

5. Galactomannan Assay Obtained from BIo-Rad:

The Platelia Aspergillus AG assay from Bio-Rad (Catalogue No. 62794) was carried out in parallel according to manufacturer's instructions.

6. Results

Using the conventional galactomannan assay (Bio-Rad), 20 out of 38 patients were identified as positive (sensitivity: 53%). Eight patients were misdiagnosed. Using the assay according to the present invention, 25 out of 38 patients were diagnosed correctly (sensitivity: 66%). Only two patients were misdiagnosed. 78 out of 79 healthy subjects were tested negative (specificity: 98.7%).

### Example 2

In separate study, it was investigated at which time point following infection the two assays yield correct positive results.

Several samples per week were collected from patients suffering from leukemia and a probable aspergillosis and analyzed as in Example 1. All these patients were eventually tested positive using assays, the conventional assay and the assay according to the present invention.

Using the assay according to the present invention, 3 patients could be diagnosed earlier using the assay according to the present invention. The delay until the conventional galactomannan assay yielded positive results was four to 29 days.

Only in one case yielded the conventional assay a positive result before the assay according to the present invention did.

### Conclusions:

The assay according to the present invention, when compared to the conventional assay using sera from patient suffering from a variety of diseases, is superior in terms of sensitivity and time until a correct positive result may be obtained.

### Example 3

A reference panel made from human plasma spiked with increasing concentrations of Aspergillus fumigatus extract, five negative samples from healthy blood donors and five samples tested using the BioRad Galactomannan Assay were subjected to the assay described in Example 1.

The results are shown in Table 1.

| | | with boiling sample | |
|---|---|---|---|
| Cut-Off > 0.5 | ID # | preparation step | without boiling step |
| Reference | RP1 | 0.23 | 6.89 |
| panel | RP2 | 0.14 | 1.10 |
| | RP3 | 0.15 | 4.06 |
| | RP4 | 1.46 | 8.11 |
| | RP5 | 1.58 | 7.97 |
| | RP6 | 3.60 | 5.78 |
| | RP7 | 5.67 | 6.69 |
| | RP8 | 8.78 | 5.92 |
| negative | Nr. 3 | 0.14 | 1.41 |
| sample from | Nr. 4 | 0.20 | 0.97 |
| healthy blood | Nr. 5 | 0.14 | 0.12 |
| donors | Nr. 9 | 0.17 | 0.15 |
| | Nr. 10 | 0.15 | 0.19 |
| positive | Nr. 6 | 3.75 | 3.31 |
| samples | Nr. 17 | 0.77 | 1.87 |
| | Nr. 25 | 0.96 | 1.54 |
| | Nr. 27 | 1.76 | 0.33 |
| | Nr. 30 | 0.91 | 1.06 |

The cut off value was 0.5, i.e. signals <0.5 indicate a negative result, signals >0.5 a positive result. Wrong results obtained without boiling step are in bold.

The reference panel comprises processed human plasma without antigen (RP1), CPD-Plasma of two different human blood donors without antigen (RP2, RP3), processed human plasma with 0.08 ng/ml Aspergillus f. lysate (RP4), processed human plasma with 0.12 ng/ml Aspergillus f. lysate (RP5), processed human plasma with 0.18 ng/ml Aspergillus f. lysate (RP6), processed human plasma with 0.5 ng/ml Aspergillus f. lysate (RP7), processed human plasma with 0.6 ng/ml Aspergillus f. lysate (RP8).

Basically the assay according to the present invention shows increasing signals in line with the increasing concentration of antigen in the samples. By contrast, results are scattered and appear to be arbitrary if the boiling step is omitted at low concentrations, showing that the boiling step helps to avoid false positive results at lower concentration of the antigen, i.e. increases the specificity of the test.

Two of the negative samples are false positive if processed without the boiling step, but are correctly evaluated as negative if the assay according to the present invention including the boiling step is used. This corroborates the finding that the specificity is to increased.

One out of five samples appears to be false negative if the boiling step is omitted, but not if the boiling step is included.

Overall, these data show that denaturing the sample increases the diagnostic reliability, in particular the specificity, and reproducibility of the assay.

### Example 4

In separate study, it was investigated at which time point following infection the two assays yield correct positive results, again, using a larger number of patients. Again, the JF5 assay was used based on the protocol described in Example 1, and the GM assay was used according to the manufacturer's instructions.

A cohort of 31 patients diagnosed as positive or probably positive according to the medical guidelines were subjected to testing on multiple days. In addition to a sample on day 0 (time of the diagnosis), additional samples prior to or after day 0 were analyzed.

Among 11 out of 31 patients, the assay based on JF5 yielded (correct) positive results over a longer time period. For eight patients, a positive diagnosis could be accomplished using the JF5-based assay at an earlier time compared to the GM assay. For four patients, the test result remained positive for a longer period of time after day 0.

By contrast, in the case of only three patients out of 31 a correct positive result could be obtained in a longer time period using the GM test compared to the test based on JF5. Two of these patients could be diagnosed as positive for a longer time after day 0, whereas one patient could be diagnosed as positive earlier before day 0 compared to the result of the JF5-based assay.

In summary, these results show that a longer time period is available for diagnosing patients correctly as positive if the JF5-based assay is used rather than the GM-based assay. Using JF5, both the time until a positive result can be obtained is shorter and the period until a positive result can no longer be obtained is extended.

1. A method for diagnosing an Aspergillus infection in a patient, said method comprising:
contacting a sample from said patient with a first antibody binding specifically to an antigen which is based on beta-1,5-galactofuranose from mycelium of a pathogenic Aspergillus strain, and
detecting said antigen in said sample,
wherein any antigen bound to said first antibody is detected by way of a non-membrane based immunoassay.

2. A method for increasing the diagnostic reliability of an immunoassay for the diagnosis of an Aspergillus infection in a patient, said method comprising:
contacting a sample from said patient with a first antibody specifically binding to an antigen based on beta-1,5-galactofuranose from mycelium of a pathogenic Aspergillus strain, and
detecting of any antigen bound to said first antibody by way of a non-membrane based immunoassay.

3. The method according to claim 1, wherein the immunoassay is an ELISA.

4. The method according to claim 1, wherein the immunoassay is a bead-based assay.

5. A method, comprising:
coating a diagnostic device with a first antibody binding specifically to beta-1,5-galactofuranose from mycelium of a pathogenic Aspergillus strain,
wherein the diagnostic device is a non-membrane based device.

6. The method according to claim 1, wherein the first antibody is coated on a microplate or a bead.

7. The method according to claim 1, wherein the first antibody is a monoclonal antibody.

8. The method according to claim 1, wherein the first antibody is the antibody JF5.

9. The method according to claim 1, wherein the Aspergillus strain is selected from the group consisting of A. fumigatus, A. flavus, A. niger, A. terreus and A. nidulans.

10. The method according to claim 1, wherein the Aspergillus strain is A. fumigatus AF 293.

11. The method according to claim 1, wherein the sample is selected from the group consisting of a serum sample and a bronchoalveolar lavage sample.

12. The method according to claim 1, wherein the method is for diagnosing an invasive aspergillosis.

13. The method according to claim 1, wherein the sample is subjected to a denaturing step prior to contacting the sample with the antibody.

14. The method according to claim 13, wherein the denaturing step is a boiling step.

15. The method according to claim 1, wherein the antigen is detected using a second antibody.

16. The method according to claim 15, wherein the second antibody is the same as the first antibody.

17. The method according to claim 2, which increases the specificity for detecting the antigen in a human patient treated with an anti-fungal drug.

18. The method according to claim 2, which increases the diagnostic reliability at an early stage of the Aspergillus infection in the patient.

19. The method according to claim 2, wherein any antigen bound to said first antibody is detected by way of a non-membrane based immunoassay, selected from the group comprising an ELISA and a bead-based assay.
